(19) United States
(12) Patent Application Publication
(10) Pub. No.: US 2024/0081643 A1
(43) Pub. Date: Mar. 14, 2024

(54) Slit Lamp angular measurement system apparatus and method thereof

(71) Applicant: Behzad BarazandehNoveyri, Mashhad (IR)

(72) Inventor: Behzad BarazandehNoveyri, Mashhad (IR)

(21) Appl. No.: 18/459,324

(22) Filed: Aug. 31, 2023

Related U.S. Application Data
(60) Provisional application No. 63/402,527, filed on Aug. 31, 2022.

(51) Int. Cl. A61B 3/135

(52) U.S. Cl. CPC A61B 3/135

(57) ABSTRACT

The present disclosure provides a device and a method for measuring angles over cornea of a patient using a simple Slit Lamp. The ophthalmic system can be used for determining desired angular orientation for ocular implants or incisions. For example, it can be used for aligning Toric IOL to desired angular orientation.

The present application claims priority of U.S. provisional patent application No. 63/402,527 filed on Aug. 31, 2022, the specification of which is hereby incorporated by reference. the specification of which is hereby incorporated by reference.

### FIELD OF INVENTION

This invention relates in general to Ophthalmic Instruments and more specifically to slit lamps.

### BACKGROUND

This section is intended to provide a background or context to the invention that is recited in the claims. The description herein may include concepts that could be pursued but are not necessarily ones that have been previously conceived or pursued. Therefore, unless otherwise indicated herein, what is described in this section is not prior art to the description and claims in this application and is not admitted to be prior art by inclusion in this section.

Slit lamp is an instrument consisting of a high-intensity light source that can be focused to shine a thin sheet of light into the eye. The lamp facilitates an examination of the anterior segment and posterior segment of the human eye, which includes the eyelid, sclera, conjunctiva, iris, natural crystalline lens, and cornea. A binocular slit-lamp examination provides a stereoscopic magnified view of the eye structures in detail, enabling anatomical diagnoses to be made for a variety of eye conditions.

A second, hand-held lens can be used to examine the retina. However, slit lamps typically don't provide accurate and reliable angular measurement of the patient eye and have to be used conjointly with other electronic devices which makes it both expensive and somehow impractical as the physician would not be able to measure and mark the angle accordingly.

Briefly described, the angular measurement of or on the eye may be needed in preparation for an ophthalmic procedure, during the procedure to confirm the accuracy of the steps being taken by the surgeon, or as a part for the post operation services a patient receives. For example, in Toric IOL implant (which refer to astigmatism correcting intraocular lenses used at the time of cataract surgery to decrease post-operative astigmatism), the angular location of the phaco incision has to be measured to prepare the patient for many ophthalmic procedures.

However, the regular slit lamps do not provide an accurate and reliable angular measurement and the devices used for this purpose are typically expensive and complicated.

Furthermore, the accuracy of such measurement is quite important. for example, it has been estimated that “for every degree of misalignment, about 3 percent of the lens cylinder power is lost,” [Toric IOLs: Nailing The Alignment, Retrieved: https://www.reviewofophthalmology.com/article/toric-iols-nailing-the-alignment], therefore a 30-degree misalignment would actually result in an increase of the patient's postoperative astigmatism.

As mentioned before, the inaccurate measurement of the angle of Toric lenses inside the eye reduces the effect of Toric intraocular lenses and ultimately leads to a decrease in the patient's vision. This may result in the need to correct the position of the lens after the operation, so that the patient's vision reaches its maximum, and otherwise, it causes the need for glasses.

On the other hand, the existing measurement methods are time consuming, which excludes the physician's possibility of performing a higher number of surgeries due to the interruption in the operation time.

Therefore, there exists a need for a simple device that can be used along with a slit lamp to provide angular measurements of or on the eye of the patient.

Furthermore, there exists a need for a device and methods capable of accurately determining the axis of cylindrical refractive power in the cornea to which the Toric IOL implant should be aligned. In addition, there is a need for systems and methods that allow for more accurate positioning of the Toric IOL implant at the optimal angular orientation for correction of the astigmatic power of the cornea.

### SUMMARY

The present disclosure provides, among others, solutions addressing the above-mentioned problems with the existing systems. This patent application provides complementary improvements that may be applied separately or in combination.

This present disclosure provides a solution to above-mentioned problems by providing an apparatus and method that can be used by the ophthalmologist or other health providers in different settings such as an operating room to accurately measure the axis of cylindrical refractive power in the cornea to which the Toric IOL implant should be aligned and therefore insert the lens into the eye. The suggested solution resolves the existing problem with most existing slit lamps which is their inability to monitor the angle of the corneal arc by conventional slit lamp devices. This invention is related to the accurate measurement of the angle of the corneal arc in the slit lamp device, which can be a mechanism to determine the exact position of the intraocular lens (Toric).

The present disclosure provides a device and method for accurately measuring slit light angel on a patient's eye without need of complicated electronic devices. The device can be integrated part of slit lamp or used as an add-on to retrofit existing slit lamps.

In one broad aspect, the present disclosure provides a slit light angle measurement system for use with a slit lamp. The system comprises a first member for connecting to a slit beam rotation mechanism of said slit lamp using a connecting member, wherein changes in an angle of a slit beam of said slit lamp creates a rotation in said first member corresponding to said changes of said angle in the slit beam; a second member for connecting to a stationary part of the slit lamp at a proximity of said first member; a sensor connected to one of said first member and second member to measure relative rotation of said first member and second member and providing an output signal corresponding to said rotation of the first member; a controller for receiving said output signal and determining an angle of said slit light from a reference point.

In some embodiments, the system may further comprise a transceiver for sending said output signal to an end device and wherein said controller is a controller of said end device.

In some embodiments, the system may further comprise a housing receiving and providing pivotal connection between said first member and said sensor.

In some examples, the connecting member may comprise bearings providing said first member with pivotal movement relative to the slit lamp.

In some examples, the connecting member may comprise gears providing said first member with pivotal movement relative to the slit lamp.

In one embodiment, the system may further comprise a transceiver for communication with an interface showing said angle of the slit light. In some examples, the system may have an interface as an integrated part of the system while in other examples it may use the transceiver to communicate with an end device such as a computer or mobile device working as the interface.

In some embodiments, the controller may be the processor of an end device and wherein said user interface is an interface of said end device.

In some embodiments of the present disclosure, the sensor may be a magnetic rotary encoder registering rotation of said first member relative to the slit lamp. In one embodiment, the sensor may be a reflective sensor measuring movement of said first member relative to the slit lamp.

In some examples, the sensor may be an optical encoder and said first member comprises a slotted disk and wherein said optical sensor measures rotation of said first member relative to the slit lamp.

In one embodiment of the present system, the sensor may be a reflective sensor measuring movement of said first member.

In some examples, the sensor used in the system may be an eddy-current killed oscillator (ECKO) measuring movement of said first member. In some other examples, the sensor may be a Wiegand sensor measuring movement of said first member.

In one embodiment, the sensor used in the system may be a Hall-effect sensor measuring movement of said first member.

In another broad aspect, the present disclosure provides a method for measuring an angle of a slit beam using a slit lamp. the method includes attaching a first member to a slit beam rotation mechanism of said slit lamp, wherein changes in an angle of said slit beam create corresponding rotation in said first member; attaching a second member to a stationary part of said slit lamp; measuring a relative rotation of said first member and said second member; and determining said angle of said slit light using the relative rotation of said first member and said second member.

In some examples of present method, the measuring a relative rotation of said first member and said second member comprises attaching a sensor to one of said first member or second member.

In some examples of present method, the determining said angle of said slit light using the relative rotation of said first member and said second member comprises generating an output signal by said sensor corresponding to the relative rotation; and communicating the output signal to a controller.

In some examples of present method, the determining said angle of said slit light using the relative rotation of said first member and said second member comprises generating an output signal by said sensor corresponding to the rotary change; and transmitting said output signal to a remote device for performing said determining the angle of said slit light relevant to said angle reference point using said registered rotary position of said first member.

In some examples of present method, the measuring a relative rotation of said first member and said second member comprises measuring changes caused by said first member in a magnetic field using a magnetic sensor.

In some examples of present method, the measuring a relative rotation of said first member and said second member comprises attaching a magnetic rotary encoder to said first member and registering rotation of said first member relative to the slit lamp.

In some examples of present method, the measuring a relative rotation of said first member and said second member comprises measuring movement of said first member using an optical sensor as said rotary position sensor.

### BRIEF DESCRIPTION OF THE DRAWINGS

The present examples will be better understood with reference to the appended illustrations which are as follows:

FIG. 1 shows a schematic perspective view of a slit lamp as known in the art.

FIG. 2(a) shows a schematic partial perspective view of a graduated scale used for calculation of slit inclination available in a typical slit lamp.

FIG. 2(b) is a corresponding photo showing the graduated scale used for calculation of slit inclination available in a typical slit lamp.

FIG. 3 illustrates a schematic view of a narrow beam light of the slit lamp in the state of crossing the coaxially sighted corneal light reflex (“CSCLR”).

FIG. 4 illustrates Coaxial illumination on an eye of a patient when front light of the slit lamp hits the eye surface perpendicular to the eye plane.

FIG. 5 illustrates a schematic view of illustrates how narrow slit beam can be placed in different angles and if it crosses CSCLR it can be used to find different meridian of the cornea.

FIG. 6(a) illustrates schematic plane view of cornea when Toric IOL axis is not aligned with cylinder (astigmatism) axis of cornea.

FIG. 6(b) shows schematic plane view of cornea when two axes are aligned.

FIG. 7(a) shows the angle measurement system wherein the angle measurement system has replaced the graduate scale (used in common slit lamps to calculate slit inclination during rotation) of a slit lamp known in the art.

FIG. 7(b) shows exploded perspective view of the angle measurement system of FIG. 7(a) in accordance with an embodiment of the present disclosure.

FIG. 8(a) shows a photo of an angle measurement system in accordance with an embodiment of the present disclosure.

FIG. 8(b) shows perspective view of the angle measurement system shown in FIG. 8(a) with a first member for connecting to a slit beam rotation mechanism of a second member for connecting to a stationary part of the slit lamp.

FIG. 9 shows exploded view of the angle measurement system shown in FIGS. 8(a) and 8(b).

FIG. 10 shows a perspective view of the connection mechanism of the angle measurement system connecting to a stationary part of the slit lamp according to one embodiment of present disclosure.

FIG. 11 shows a detailed view of the connection mechanism shown in FIG. 10.

FIG. 12 shows an exemplary magnetic rotary encoder sensor used in the angle measurement system in accordance with one embodiment.

FIG. 13 shows the pin connections of the magnetic rotary encoder sensor shown in FIG. 12.

FIG. 14 illustrates a chart showing the position error within a signal period for an exemplary sensor used in the angle measurement system.

FIG. 14 illustrates a chart showing the position error within a signal period for an exemplary sensor used in the angle measurement system.

FIG. 15(a) illustrates the position error within a signal period for an exemplary sensor used in the angle measurement system.

FIG. 15(b) illustrates an alternative chart showing the position error within a signal period for an exemplary sensor used in the angle measurement system.

FIG. 16 shows a flowchart showing the information received from the sensor and processing applied to it in accordance with one embodiment of present disclosure.

FIG. 17 shows the steps involved in the method used for measuring angle rotation in accordance with one embodiment of the present disclosure.

FIG. 18 illustrates the front view of a housing having an attachment mechanism for connecting the system to a slit lamp in accordance with one embodiment the present disclosure.

FIG. 19 illustrates the perspective view of the housing shown in FIG. 18.

FIG. 20 illustrates the top view of the housing shown in FIG. 18.

FIG. 21 illustrates the side view of the housing shown in FIG. 18.

FIGS. 22 and 23 illustrate the housing shown in FIG. 18 as mounted on a slit lamp.

FIG. 24 illustrates an exemplary design of a PCB design for use with the controller and processor of the system in accordance with one embodiment of the present invention.

FIG. 25 illustrates an exemplary electronic schematic for use with the controller and processor of the system in accordance with one embodiment of the present invention.

FIG. 26 illustrates an application interface for use with the system in accordance with one embodiment of the present invention.

### DETAILED DESCRIPTION

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

Moreover, the described features, structures, or characteristics of the invention may be combined in any suitable manner in one or more embodiments. It will be apparent to those skilled in the art that various modifications and variations can be made to the present invention without departing from the scope of the invention. Thus, it is intended that the present invention cover the modifications and variations of this invention provided they come within the scope of the appended claims and their equivalents. Reference will now be made in detail to the preferred embodiments of the invention.

Cataracts are clouded regions of the eye that can develop in the natural crystalline lens of an eye. A cataract can range in degree from slight clouding to complete opacity. Typically, formation of cataracts in human eyes is an age-related process. If left untreated, cataracts can lead to blindness.

Different methods of surgery have been developed for the treatment of cataracts. Typically, an incision is made in the eye and the natural crystalline lens is removed. An artificial lens called an intraocular lens (IOL) is then inserted in the capsular bag of the eye in place of the natural crystalline lens. The spherical optical refractive power of the IOL implant may be selected, for example, so as to place the eye in a substantially emmetropic state when combined with the refractive power of the cornea of the eye.

A cataract surgery typically requires a phaco incision, through which the patient's natural crystalline lens is removed and an artificial intraocular lens (IOL) is inserted, to be made at the limbus of a patient's eye. In some cases, it may be advantageous for this phaco incision to be made along a meridian at some specified angular orientation on the eye. Moreover, cataract surgeries, and other ophthalmic procedures, may also involve additional angular measurements. For example, a cataract surgery may involve angular measurements so that the surgeon can align a Toric IOL to a particular angular orientation. Toric IOL implants provide a degree of correction for any regular astigmatism which may exist in the patient's cornea. Such regular astigmatism results from a difference in the degree of curvature of the cornea in orthogonal meridians such that the eye has different focal lengths for light incident upon it in each of the planes of the two orthogonal meridians. Regular astigmatism has an axis associated with it that indicates its angular orientation.

A Toric IOL implant has spherical and cylindrical refractive power. The cylindrical refractive power of the Toric IOL more effectively corrects corneal astigmatism if it is accurately aligned with the angular orientation of the corneal astigmatism of the patient's eye. Therefore, the angular location of the phaco is important.

An ophthalmic surgery may also involve Limbal Relaxing Incisions (LRI) to be performed in an effort to correct corneal astigmatism. Even once the desired angular locations for these incisions have been determined, the surgeon may still need some means for actually identifying the desired angular locations on the eye. Other ophthalmic procedures may also involve certain angular measurements of the eye to be made.

Similarly, in postoperative period after lamellar or penetrating keratoplasty when there is a need to remove sutures to reduce corneal graft toricity, in “suture on state”, as well as in “suture off state” when making incision corresponding to steep meridian or axis (corneal astigmatism) in different angles angle measurement may be required.

However typical slit lamps, as one of most popular instruments, known in the art either don't provide angular measurement or this measurement is not accurate enough for the above-mentioned purposes.

By way of summary, slit lamp is an instrument used routinely in every ophthalmic examination. It generally directs light as a narrow beam on the eye a patient, from various directions, which allows seeing eye coaxial with visual axis of the patient. When this narrow beam is not coaxial, the viewer can see an optical section of the eye structures as far as light can penetrate including cornea, anterior chamber, lens and anterior vitreous.

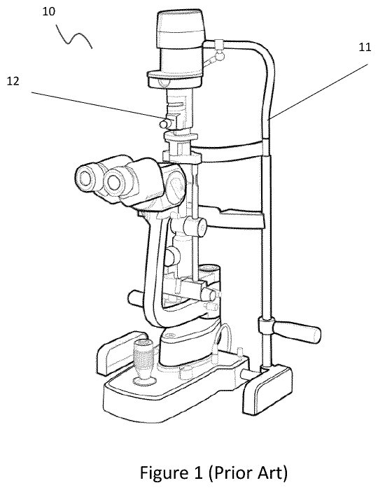

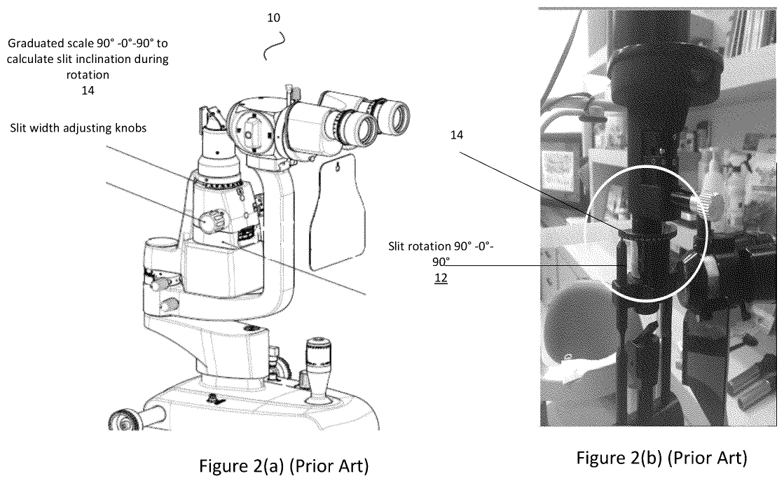

The typical slit lamp 10, as shown in FIG. 1, include a manual measuring means 14 used in combination with narrow slit beam rotation member 12, as shown in FIGS. 2(a) and 2(b) for determining the angular orientation of the beam on the cornea with steps of 5 to 10 degrees. This is primarily not easy to read and secondary the angle provided by manual measuring means is not accurate.

The present disclosure provides a slit beam angle measurement system by adding the invented device to a slit lamp either as an original part of the device during manufacturing or as a retrofit for existing slit lamps. Typically, the accuracy of such system in determining axis of corneal astigmatism may be increased to up to tenth of a degree as like as corneal topography.

In one example, the present disclosure provides a system and method for the measurement of the cylindrical power and axis of a patient's cornea by corneal topography with an accuracy of one tenth ( 1/10) of a degree.

The topography may measure corneal astigmatism in patients. The corneal astigmatism is the stand-alone source of refractive astigmatism in aphakic (no lens state of the eye) and main source of astigmatism in phakic eyes. Therefore, it is important to avoid any discrepancy between the topography and the Slit Lamp's measurement of cornea. Therefore, it is important to find the correct axis to be treated by Toric IOL accurately as it has been known in the that, for every 3 degrees of deviation from the predetermined axis, the IOL loses 10% of its astigmatism correction power. A rotation of 30 degrees from the programmed axis can result in loss of 100% of the astigmatism correction. For example, a method for aligning the astigmatic axis of a Toric IOL with the astigmatic axis of the cornea of a patient's eye during cataract surgery includes determining at first magnitude and axis of the corneal astigmatism; removing the natural lens; inserting a Toric IOL, deliberately misaligning the astigmatic axis of the Toric IOL by at least 15° from the first astigmatic axis of the cornea; and intra-operatively measuring the total refractive power of the pseudophakic eye while the Toric IOL is deliberately misaligned.

Another method for aligning the astigmatic axis of a Toric IOL with the astigmatic axis of the cornea of a patient's eye during cataract surgery includes with slit lamp a long narrow beam is created on cornea under coaxial illumination (viewing arm in line with illuminating arm crossing the corneal light reflex) then narrow beam aligned with the corneal astigmatism axis determined by topography then marking the cornea at the determined axis in sitting position. Then removing the natural lens and inserting a Toric IOL, Deliberately misaligning the astigmatic axis of Toric IOL by at least 15° from the astigmatic axis of the cornea, rotating the lens to align the astigmatic axis of the Toric IOL with cornea astigmatic axis.

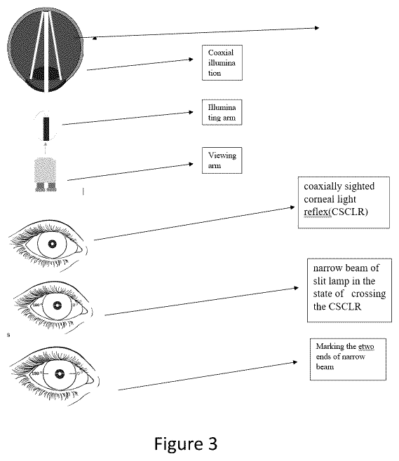

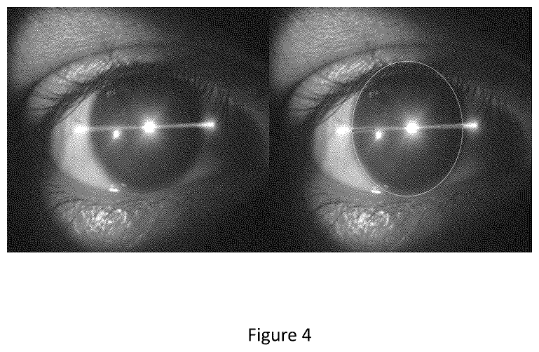

FIG. 3 shows the eye of a patient with narrow beam of slit lamp in the state of crossing the coaxially sighted corneal light reflex (“CSCLR”). This reflex is the center of choice in all refractive procedures because its location is at intercept of visual axis and cornea. FIG. 4 shows Coaxial illumination. When front light hits the object surface perpendicular to the object plane, we speak of coaxial illumination. Coaxial illumination can additionally be collimated, i.e., rays are parallel to the optical axis (within a certain degree in coaxial illumination light hits the corneal surface perpendicular and create a point of bright reflection.

In coaxially sighted condition the viewing arm of the slit lamp is in the line of illuminating arm. If these conditions are done simultaneously and, in this condition, if the patient asked to see the bright light of the slit lamp the viewer eye and patients' eye are on the same axis now the specular bright corneal reflex is called coaxially sighted corneal light reflection. It is the center of choice in all refractive procedures because its location is at intercept of visual axis and cornea.

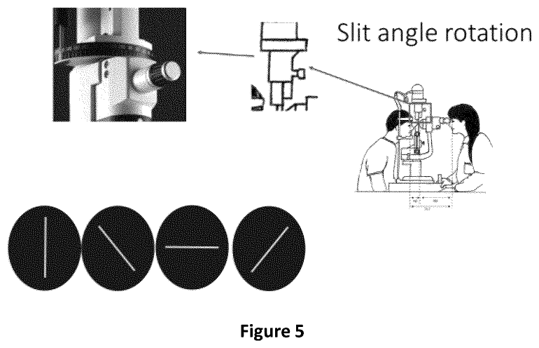

FIG. 5 shows narrow slit beam can be placed in different angles and if it crosses CSCLR it can be used to find different meridian of the cornea.

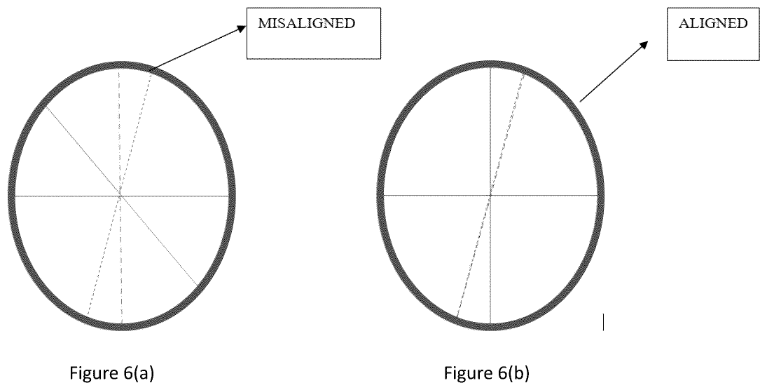

FIG. 6(a) shows a situation illustrating a plane view of cornea when Toric IOL axis is not aligned with cylinder (astigmatism) axis of cornea. Toric IOL axis is at 90 and corneal cylinder axis is at 80 degrees. Even if IOL power was correct, residual astigmatism would occur and astigmatism persists. Suppose if Toric IOL cylinder=−2 D and corneal cylinder is =2.5 D. The residual cylinder in this condition is 0.7 at 130. Therefore, the misalignment not only has increased the amount of residual astigmatism but also axis of residual astigmatism is different from axis of Toric IOL cylinder and corneal cylinder axis here at 130 degree.

FIG. 6(b) illustrate a situation in which the plane view of cornea when two axes are aligned. Dotted line represents cylinder axis of cornea at 80 axis and dashed line represents Toric lens axis .in this situation if the power of Toric IOL is correct it neutralizes corneal cylinder and astigmatism disappear in patient refraction.

Corneal astigmatism power and axis can be estimated by a corneal topographer or keratometer at upright position. This position is necessary to avoid cyclotorsion of the eye that may happen in supine position. Then Toric IOL power and orientation is calculated by Toric IOL calculator. Surgeons can proceed to cataract surgery. There are a variety of methods to align Toric IOL axis with cornea cylinder axis.

The conventional method has three steps exist: when the patient is at sitting position, with a horizontal bubble level 0-180 axis (meridian) of the cornea is marked. Then intraoperative in the supine position surgeon use a device with angular graduation (such as mendez ring) to find and mark the limbus at the angular axis calculated by Toric IOL calculator (target axis). After removing natural lens and inserting Toric IOL, surgeon align Toric IOL axis to the estimated axis of the corneal astigmatism (target axis) using this mark at limbus.

Mobile app uses the inclinometer available on the hardware of the smartphone to obtain a properly aligned image in upright position. This facilitates finding 0-180 axis (meridian) of the cornea then intraoperative in supine position surgeon use a device with angular graduation (such as Mendez ring) to find and mark the limbus at the angular axis calculated by Toric IOL calculator. After removing natural lens-inserting Toric IOL surgeon align Toric IOL axis to the estimated axis of the corneal astigmatism (target axis) using this mark at limbus.

Basically, in these two methods three steps exist none of them uses CSCLR as a reference point for IOL centration.

There also exists alternative methods of axis marking: Iris fingerprinting and digital overlay:

In iris fingerprinting in which an image was captured when the patient was initially examined with a dilated pupil using a high-definition camera mounted on the slit lamp. Software determined the horizontal and vertical meridians, and an overlying protractor was superimposed that allowed the surgeon to detect the number of degrees at which each crypt, pigment, vessel, or stromal pattern was located. During operation surgeons use these landmarks to align IOL to the right angle.

Digital overlay: Each of these technologies captures a preoperative image by an imaging device specific for each of them. The information Is stored on a USB drive that is registered at the beginning of surgery on the microscope equipped with each technology. With the microscope, a then superimposed “live” on the eye during surgery the surgeon could visualize three parallel lines (Cellist) or one line (verion) that represented the target meridian to which the Toric lens was later rotated. The information transferred to the microscope .during operation a then superimposed “live” on the eye during surgery to identify the target meridian on either a monitor or through the oculars on the operating microscope the pros and cons of each method are listed in the following tables 1 and 2.

TABLE 1

| | | Reference Mark | | | |
|---|---|---|---|---|---|
| | Skill of | Upward | Fade | Smearing | |
| | Surgeon | Deviation | of Ink | of Ink | Time Consuming |
| Conventional | yes | yes | yes | yes | Three-step process |
| manual | | | | | |
| method | | | | | |
| image- | no | no | no | no | Three step and need |
| guided | | | | | of Pre and |
| systems | | | | | intraoperative image |
| | | | | | capture |
| Slit lamp | no | no | no | no | Two step and rapid |
| assisted | | | | | |
| angular | | | | | |
| measurement | | | | | |
| system | | | | | |

TABLE 2

| | Reference | Reference Point Is | | Need to Be |
|---|---|---|---|---|
| | Point Is | Geometric Center or | Intraoperative | Backed Up by |
| | CSCLR | Pupil Center | Drift | Another Method* |
| image-guided | no | yes | yes | yes |
| systems | | | | |
| Slit lamp assisted | yes | no | no | no |
| angular measurement | | | | |
| system. | | | | |

*(possibility that registration can fail either at the beginning of the procedure or during the operation)

Narrow beam of slit lamp is set on the target meridian calculated previously (here is 0-180). Then two mark on the periphery of cornea are made by a fine needle following the light streak of narrow slit beam in cornea periphery. Position or alignment of Toric IOL can be seen intraoperative relative to these marks. After operation also narrow slit beam positioned in the intended axis can be used for finding relative position of actual axis meridian of Toric IOL.

In all these stages the position of narrow slit beam can be displayed on the mobile or computer display by using an app designed for each of them. The information relayed from the instrument wirelessly to the software in mobile app or desktop computer.

Alternatively, the position of narrow slit beam can be transferred to ocular of the microscope by a transceiver.

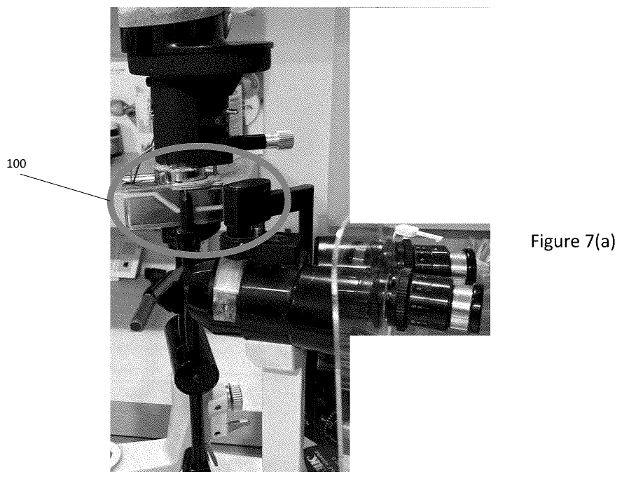

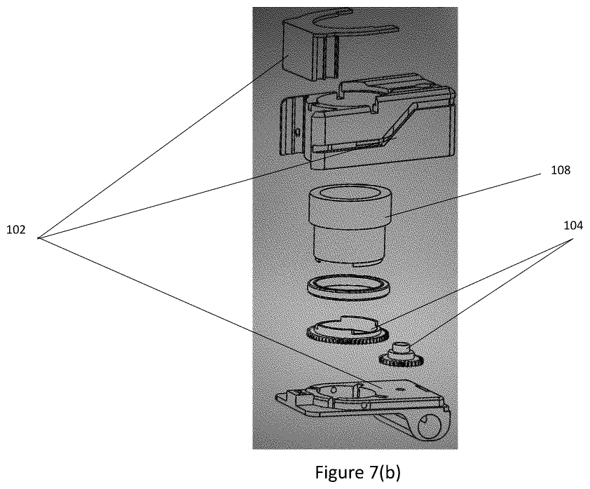

FIG. 7(a) shows an embodiment of the angle measurement device 100 disclosed where in the device 100 replaces the manual measuring means 14 of the slit lamps 10. The device 100 may communicate with an external processor, interface and be connected to an external power source or have all these elements as an integrated part of the device 100.

It will be appreciated by those skilled in the art that the device 100 can be an integrated part of a new device or be used to retrofit an existing slit lamp.

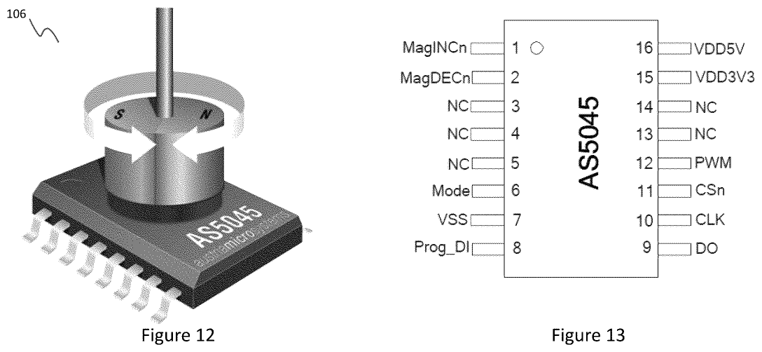

FIG. 7(b) shows details of device 100 in an exploded view. As illustrated a number of elements may form the housing 102 which receives the sensor (not shown here). A mechanism 108 allows the device 100 to rotate with the slim bean rotation mechanism 12 relative to a fixed part of the slim light 10 and transfer this rotation via the gears 104 to a rotation measurable by a sensor 106 an example of which is shown in FIG. 12.

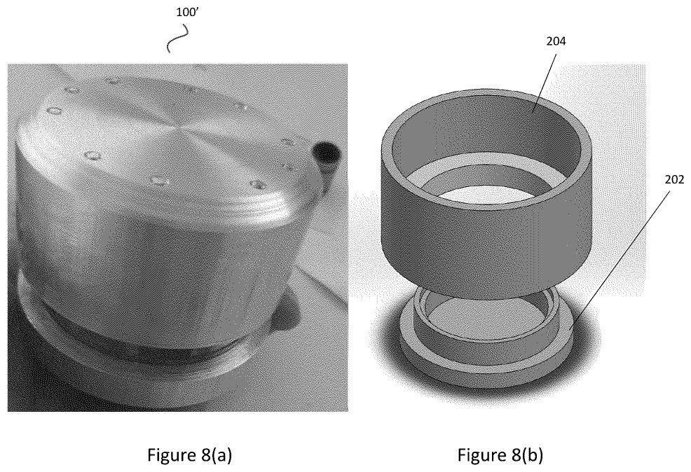

FIG. 8(a) shows the angle measurement device 100′ disclosed in accordance with an alternative embodiment of the present disclosure where in the device 100′ connects to the top of the slim bean rotation mechanism 12. The connection may be done via any means known in the art including pins, screws or adhesive material.

Like other embodiments the device 100′ may communicate with an external processor, interface using an integrated transceiver and be connected to an external power source or have all these elements as an integrated part of the device 100.

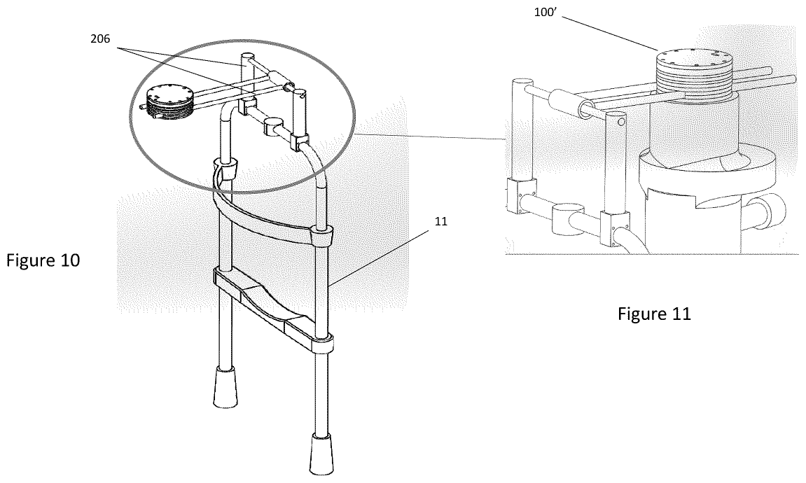

FIG. 7(b) shows details of the device 100′. As illustrated a number of elements may form the housing 204 which receives the sensor (not shown here) and other elements including all the circuits, and possibility, a power source. As shown in FIGS. 10 and 11 one of the members 202 and 204 may connect to a fixed part, such as the member 11 receiving the patient's head and connected to the table, of the slit lamp 10 by a connecting mechanism 206 to allow the measurement of the angle of members which is connected to the slim bean rotation mechanism 12.

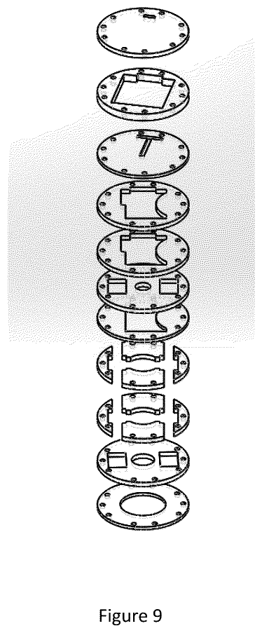

FIG. 9 shows an exploded view of the device 100′ and how different layers are designed to accommodate different elements of the angle measurement system including the circuits, battery, transceiver and the sensor.

It would be appreciated by those skilled in the art that device 100′ can be designed in any other shape and have cavities inside of it in other shapes in order to accommodate a user interface and a processor if required. Also, the power supply may be provided using a connection to the slit lamp 10 or a battery. The battery may be chargeable using wired, or wireless charging or even a photovoltaic screen on top of the device 100′ to receive light from the surroundings.

It would be appreciated that the sensor 106 may be any kind of sensor capable for measuring angles including any kind of optical or magnetic sensors including magnetic rotary encoder, reflective sensor, an optical encoder, a reflective sensor, eddy-current killed oscillator (ECKO), Wiegand sensor, and Hall-effect sensor.

It would also be appreciated that the sensor may be connected to the member attached to the fixed part of the slit lamp, the member attached to the slit beam rotation member or outside the device 100 or 100′.

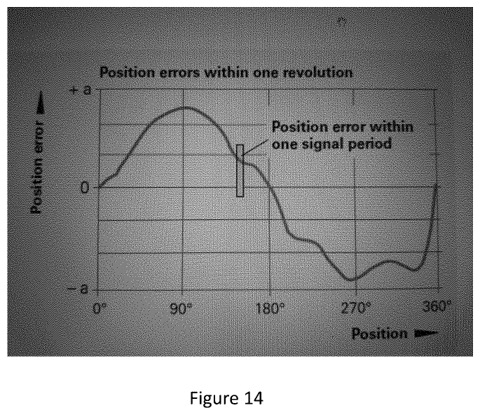

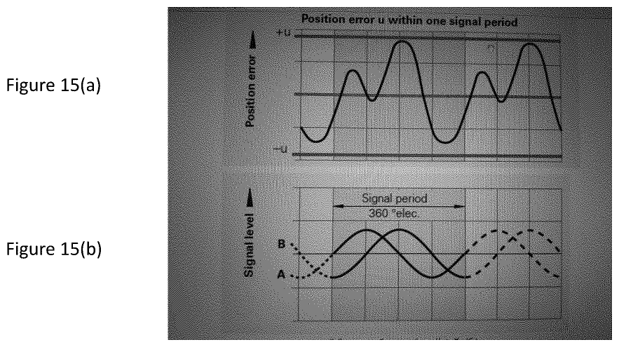

FIGS. 14, 15(a) and 15(b) show the position error in one period cycle, and for different signal levels.

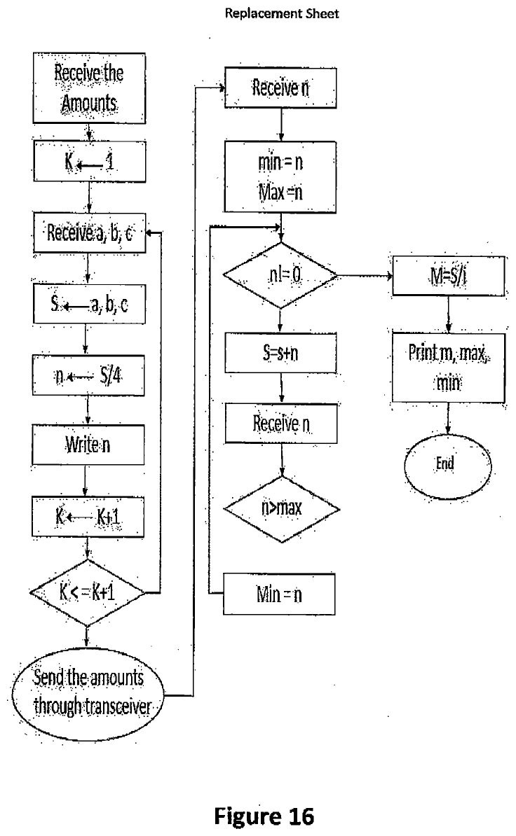

FIG. 16 shows an exemplary processing performed by a controller used in the measurement system on the data received from the sensor 106.

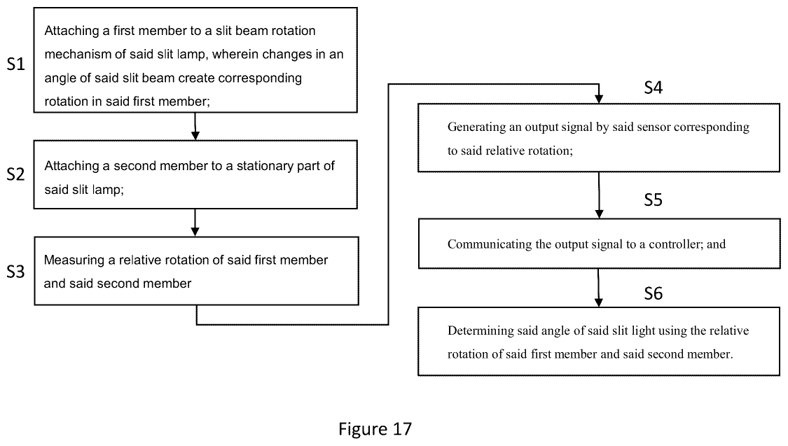

FIG. 17 shows an example of the steps required to be taken for measuring an angle of the slit lamp light. In S1, the method requires attaching a first member to a slit beam rotation mechanism of said slit lamp, wherein changes in an angle of said slit beam create corresponding rotation in said first member. S2 includes attaching a second member to a stationary part of said slit lamp. In S3 the method comprises measuring a relative rotation of said first member and said second member.

It would be appreciated by those skilled in the art that the measurement of the relative movement of the first and second member may be done using different sensors as disclosed herein. Also, the sensor may be connected to either of the members or to an external reference point.

S4 of the method includes generating an output signal by said sensor corresponding to the relative rotation. The signal then is received by a controller in S5 and is used to calculate the rotation and angle of the slit light in S6. The result can be shown on a display for use of the operator of the slit lamp.

It will be appreciated by those skilled in the art that the processing and display may be done on an end device such as a cellular phone of the operator. The system may equally have a designated independent controller and/or interface for use by the operator.

As shown in FIGS. 12 and 13, in one example, the slit light angel measurement system may have an AS5045 sensor. The AS5045 is a contactless magnetic rotary encoder for accurate angular measurement over a full turn of 360°. The AS5045 sensor is a system-on-chip, combining integrated Hall elements, analog front end and digital signal processing in a single device.

In some embodiments, as illustrated in FIG. 14, to measure the angle, only a simple two-pole magnet, rotating over the center of the chip, may be required. The magnet may be placed above or below the IC. The absolute angle measurement provides instant indication of the magnet's angular position with a resolution of 0.0879°=4096 positions per revolution. This digital data is available as a serial bit stream and as a PWM signal. An internal voltage regulator allows the AS5045 to operate at either 3.3 V or 5 V supplies.

This sensor has some benefits such as complete system-on-chip, flexible system solution provides absolute and PWM outputs simultaneously. It is also ideal for applications in harsh environments due to contactless position sensing. In some examples it may require no calibration.

Table 3 shows the description of each pin of the standard SSOP16 package (Shrink Small Outline Package, 16 leads, body size: 5.3 mm×6.2 mmm; as illustrated in FIG. 13).

Pins 7, 15 and 16 are supply pins, pins 3, 4, 5, 6, 13 and 14 are for internal use and must not be connected. Pins 1 and 2 are the magnetic field change indicators, MagINCn and MagDECn (magnetic field strength increase or decrease through variation of the distance between the magnet and the device). These outputs can be used to detect the valid magnetic field range. Furthermore, those indicators can also be used for contact-less push-button functionality. Pin 6 Mode allows switching between filtered (slow) and unfiltered (fast mode).

In some embodiments, the sensor may be a CMOS standard process and uses a spinning current Hall technology for sensing the magnetic field distribution across the surface of the chip. The integrated Hall elements are placed around the center of the device and deliver a voltage representation of the magnetic field at the surface of the IC.

Through Sigma-Delta Analog/Digital Conversion and Digital Signal-Processing (DSP) algorithms, the AS5045 provides accurate high-resolution absolute angular position information. For this purpose, a Coordinate Rotation Digital Computer (CORDIC) calculates the angle, and the magnitude of the Hall array signals.

The DSP is also used to provide digital information at the outputs MagINCn and MagDECn that indicate movements of the used magnet towards or away from the device's surface. A small low cost diametrically magnetized (two-pole) standard magnet provides the angular position information. Further details of this exemplary sensor can be found in [A55045—12 BIT PROGRAMMABLE MAGNETIC ROTARY ENCODER DATA SHEET—austriamicrosystems].

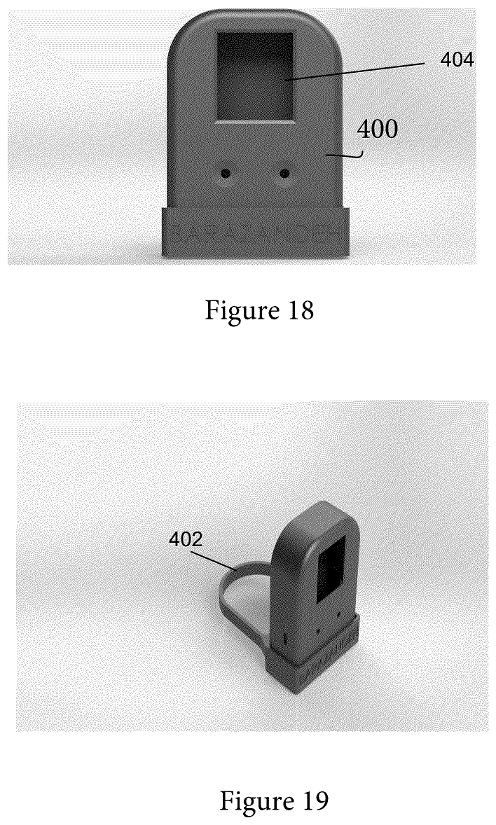

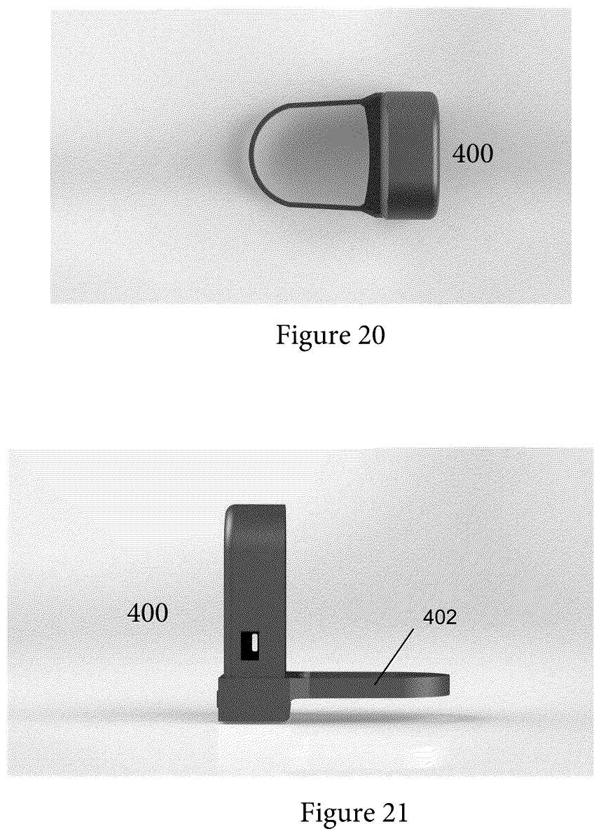

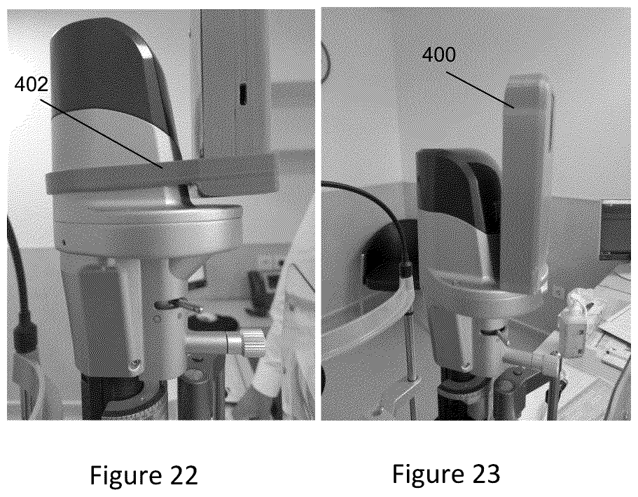

As shown in FIGS. 18 to 21, in some embodiments a housing 400 may be used to attach the device or system to a slit lamp. In some examples the housing 400 may comprise an attachment mechanism 402 for connecting the system to a slit lamp as shown in FIGS. 22 and 23. It will be appreciated by those skilled in the art that another attachment mechanism may be used to attach housing 400 to the slit lamp.

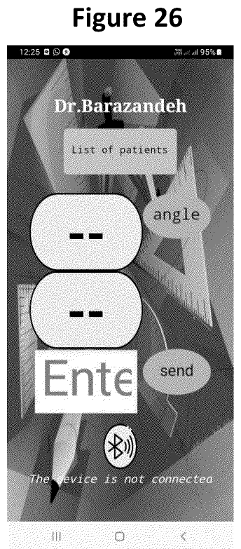

In some examples, a display (not shown) may be placed in an opening 404 allowing the device to provide the angle and other information to its user without requiring a mobile application. In some examples, a mobile application interface as shown in FIG. 26 may be used to receive the angular changes and other data form the device attached to the slit lamp.

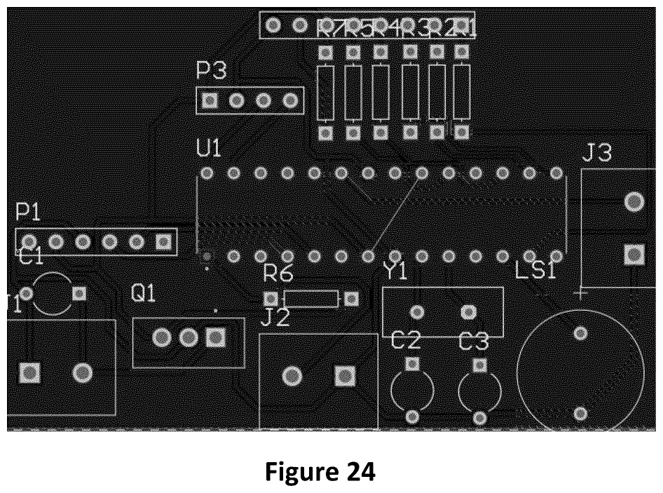

As illustrated in FIG. 24, in some examples a PCB design may be used with the controller and processor of the system in accordance with one embodiment of the present invention.

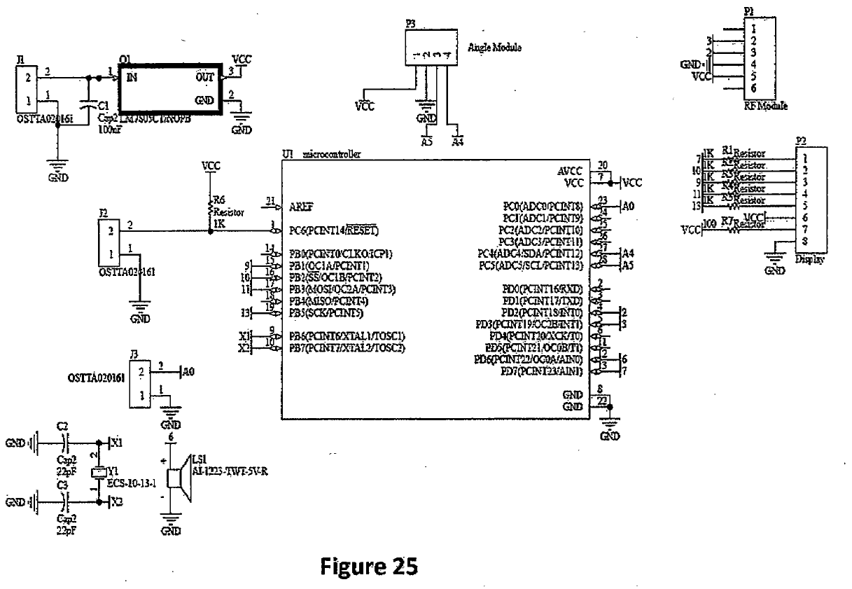

In some embodiments of the present disclosure, the system and device may have electronic system with the electronic schematic shown in FIG. 25.

| Pin | Symbol | Type | Description |
|---|---|---|---|
| 1 | MagINCn | DO_OD | Magnet Field Magnitude INCrease: |
| | | | active low, indicates a distance |
| | | | reduction between the magnet and |
| | | | the device surface. See Table 5 |
| 2 | MagDECn | DO_OD | Magnet Field Magnitude DECrease: |
| | | | active low, indicates a distance |
| | | | increase between the device and the |
| | | | magnet. See Table 5 |
| 3 | NC | — | Must be left unconnected |
| 4 | NC | — | Must be left unconnected |
| 5 | NC | — | Must be left unconnected |
| 6 | Mode | — | Select between slow (open, low: |
| | | | VSS) and fast (high) mode. Internal |
| | | | pull-down resistor. |
| 7 | VSS | S | Negative Supply Voltage (GND) |
| 8 | Prog_DI | DI_PD | OTP Programming Input and Data |
| | | | Input for Daisy Chain mode. Internal |
| | | | pull-down resistor (~74 kΩ). |
| | | | Connect to VSS if not used |
| 9 | DO | DO_T | Dala Output of |
| | | | Synchronous Serial Interface |
| 10 | CLK | DI, ST | Clock Input of |
| | | | Synchronous Serial Interface; |
| | | | Schmitt-Trigger input |
| 11 | CSn | DI_PU, | Chip Select, active low, Schmitt- |
| | | ST | Trigger input, internal put-up resistor |
| | | | (~50 kΩ) |
| 12 | PWM | DO | Pulse Width Modulation of approx. |
| | | | 1 kHz; LSB in Mode3 x |
| 13 | NC | — | Must be left unconnected |
| 14 | NC | — | Must be left unconnected |
| 15 | VDD3V 3 | S | 3 V-Regulator Output, internally |
| | | | regulated from VDD5 V. Connect to |
| | | | VDD5 V for 3 V supply voltage. Do not |
| | | | load externally. |
| 16 | VDD5 V | S | Positive Supply Voltage, 3.0 to 5.5 V |

1. A slit light angle measurement system for use with a slit lamp, the system comprising:
a first member for connecting to a slit beam rotation mechanism of said slit lamp using a connecting member, wherein changes in an angle of a slit beam of said slit lamp creates a rotation in said first member corresponding to said changes of said angle in the slit beam;
a second member for connecting to a stationary part of the slit lamp at a proximity of said first member;
a sensor connected to one of said first member and second member to measure relative rotation of said first member and second member and providing an output signal corresponding to said rotation of the first member; and
a controller for receiving said output signal and determining an angle of said slit light from a reference point.

2. The system in claim 1, further comprising a transceiver for sending said output signal to an end device and wherein said controller is a controller of said end device.

3. The system in claim 1, further comprising a housing receiving and providing pivotal connection between said first member and said sensor.

4. The system in claim 3, wherein said connecting member comprises bearings providing said first member with pivotal movement relative to the slit lamp.

5. The system in claim 3, wherein said connecting member comprises gears providing said first member with pivotal movement relative to the slit lamp.

6. The system in claim 2, wherein said transceiver communicates with an interface showing said angle of the slit light.

7. The system in claim 1, wherein said controller is a processor of an end device and wherein said user interface is an interface of said end device.

8. The system in claim 1, wherein said sensor is a magnetic rotary encoder registering rotation of said first member relative to the slit lamp.

9. The system in claim 1, wherein said sensor is a reflective sensor measuring movement of said first member relative to the slit lamp.

10. The system in claim 1, wherein said sensor is an optical encoder and said first member comprises a slotted disk and wherein said optical sensor measures rotation of said first member relative to the slit lamp.

11. The system in claim 1, wherein said sensor is a reflective sensor measuring movement of said first member.

12. The system in claim 1, wherein said sensor is an eddy-current killed oscillator (ECKO) measuring movement of said first member.

13. The system in claim 1, wherein said sensor is a Wiegand sensor measuring movement of said first member.

14. The system in claim 1, wherein said sensor is a Hall-effect sensor measuring movement of said first member.

15. A method for measuring an angle of a slit beam using a slit lamp, the method comprising:
attaching a first member to a slit beam rotation mechanism of said slit lamp, wherein changes in an angle of said slit beam create corresponding rotation in said first member;
attaching a second member to a stationary part of said slit lamp;
measuring a relative rotation of said first member and said second member; and
determining said angle of said slit light using the relative rotation of said first member and said second member.

16. The method in claim 15, wherein determining said angle of said slit light using the relative rotation of said first member and said second member comprises attaching a sensor to one of said first member or second member.

17. The method in claim 15, wherein determining said angle of said slit light using the relative rotation of said first member and said second member comprises:
generating an output signal by said sensor corresponding to said relative rotation; and
communicating the output signal to a controller.

18. The method in claim 15, wherein determining said angle of said slit light using the relative rotation of said first member and said second member comprises:
generating an output signal by said sensor corresponding to said rotary change; and
transmitting said output signal to a remote device for performing said determining the angle of said slit light relevant to said angle reference point using said registered rotary position of said first member.

19. The method in claim 15, measuring a relative rotation of said first member and said second member comprises measuring changes caused by said first member in a magnetic field using a magnetic sensor or measuring movement of said first member using an optical sensor as said rotary position sensor.

20. The method in claim 15, measuring a relative rotation of said first member and said second member comprises attaching a magnetic rotary encoder to said first member and registering rotation of said first member relative to the slit lamp.
